(19) United States
(12) Patent Application Publication
(10) Pub. No.: US 2008/0108882 A1
(43) Pub. Date: May 8, 2008

(54) Method of assessing breastfeeding

(75) Inventor: Catherine Peta Garbin, Floreat (AU)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Medela Holding AG, Baar (CH)

(21) Appl. No.: 11/903,361

(22) Filed: Sep. 21, 2007

Related U.S. Application Data
(60) Provisional application No. 60/846,464, filed on Sep. 22, 2006.

(51) Int. Cl. A61B 5/00

(52) U.S. Cl. 600/300

(57) ABSTRACT

The invention includes methods of assessing the health of a breastfeeding relationship that include clinical assessment in four categories. A first assessment involves a determination of maternal and infant contentment. A second assessment involves a determination of comfort. A third assessment involves a determination of milk production. A fourth assessment involves a determination assessing infant swallowing.

This application claims benefit of U.S. Provisional Application No. 60/846,464, filed Sep. 22, 2006.

### BACKGROUND OF THE INVENTION

1. Field of the Invention

The invention is directed to breastfeeding. In particular, the invention is directed to a tool for assessing breastfeeding. More particularly, the invention is an improved method applicable to breastfeeding, which improves breastfeeding success and provides identification of clinical problems and the likely causes of specific aspects of breastfeeding behavior.

2. Description of the Prior Art

Currently, the treatment of breastfeeding problems comprises a number of “hit and miss” methods. For example, a lactation consultant routinely observes a mother with breastfeeding problems and then picks a likely scenario or cause of the problem. If the intervention indicated by the scenario is successful at resolving the problem, the consultant needs to investigate no further. However, if the problem is not resolved, the consultant selects a second likely cause and so on. While experience-based problem solving can be successful, it tends to be subjective and results tend to vary with the investigator.

In medical disciplines, it is conventional that the investigator collects information before making a diagnosis, which then indicates a course of action. The information collecting process includes the acquisition of data by objective testing, according to one or more standardized method, in order to form a diagnosis. The current practice, with respect to investigating breastfeeding problems, is based on experience of the investigator and not typically the collection of objective evidence, hence the “hit and miss” success of interventions. If a means of clinically assessing (or testing) breastfeeding problems existed, there may be a clearer or more certain course of action suggested to the investigator, when confronted with a breastfeeding problem.

Therefore, this invention is an objective assessment tool for the diagnosis and treatment of breastfeeding problems.

### SUMMARY OF THE INVENTION

The invention, in one embodiment, is a tool usable by any competent person and in particular, for example, lactation professionals, medical professionals and the like. The tool uses objective measurements and/or assessments. The measurements allow the identification of clinical problems related to breastfeeding and the likely causes of specific aspects of breastfeeding behavior. These aspects can then be targeted for corrective intervention.

In a most general form, the invention includes a clinical assessment in four categories. A first assessment involves a determination of maternal and infant contentment. A second assessment involves a determination of comfort. A third assessment involves a determination of milk production. A fourth assessment involves a determination assessing infant swallowing.

The method can advantageously be used by educators to structure antenatal breastfeeding classes, mothers and partners to plan their own breastfeeding care, hospital workers and other health care professionals, community health care workers and breastfeeding products sales teams.

This, and additional objects, advantages, features and benefits of the invention will become apparent from the following specification.

### BRIEF DESCRIPTION OF THE DRAWINGS

FIG. 1 is a table which illustrates a method according to an embodiment of the invention.

### DETAILED DESCRIPTION OF AN EMBODIMENT

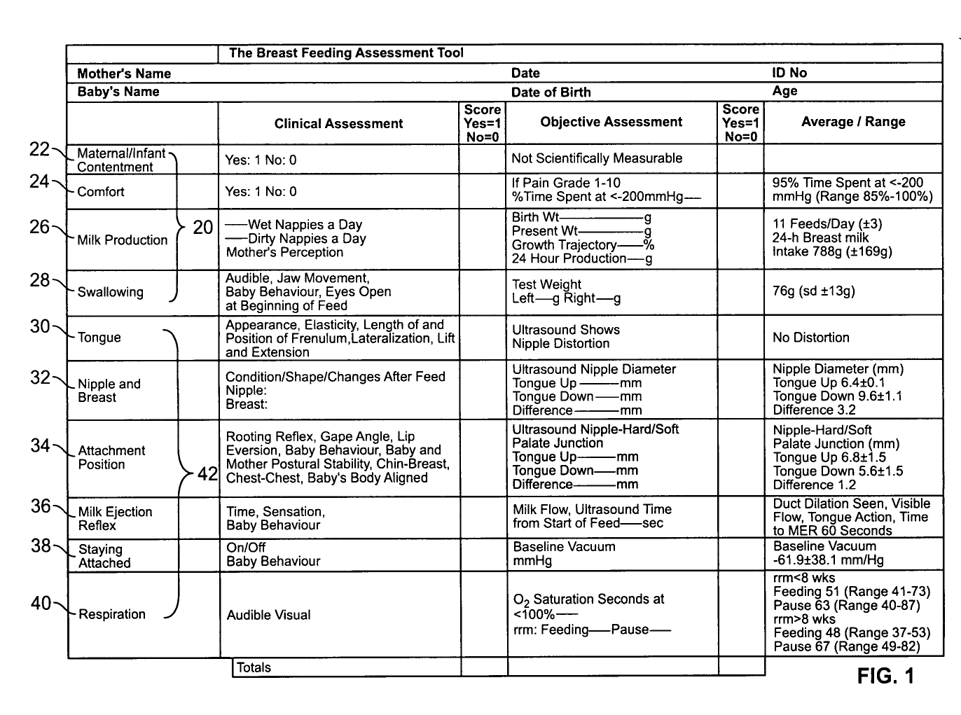

Referring to the drawings, FIG. 1 shows a table summarizing the four main components of the method according to one embodiment of the invention. FIG. 1 shows a tool 20, including a first assessment component 22 related to baby/maternal contentment. A second assessment component 24 is related to whether the mother is comfortable. A third assessment component 26 is related to the milk production of the mother. A fourth assessment component 28 is related to the swallowing of the infant.

The assessment tool 20, including components 22-28 may be used without any associated tests, such as the mother in a hospital making a self-assessment using the four components. If she answers “no” to any of the four assessment components, she should seek help.

Also, a nurse in a hospital can use the tool 20 in the form of the above-described 4 component assessment to make a general assessment of breastfeeding of a patient. Should her patient answer “no” to any of the 4 assessment components then she should follow up with a more extensive assessment, which may involve more detailed questioning directed to the component which generated the “no” response and further, may elect to order further testing and/or observation, for example, or the suggestion to consult with a lactation professional.

One example of a method according to the invention includes presenting to the subject a diagnostic survey, which is in an oral or in the alternate, a written form. A first assessment question is asked, which is similar or identical to, “Are you and your baby content?” If the answer is “no,” one or more follow up question is asked to determine the cause of the lack of contentedness. For example, if the mother is wondering if her milk supply is sufficient, a milk production assessment is performed to determine the amount of milk generated each feeding in a 24 hour period. In response to the answer to the follow up question, a treatment plan is put into place addressing the cause. If the answer is “yes,” a second assessment question is asked.

The second assessment question is asked, which is similar or identical to, “Is the feeding comfortable for you and your baby?” If the answer is “no,” one or more follow up question would be asked to determine the cause of the lack of comfort. For example, a pain grade may be determined (a common practice for assessing pain in increments, such as from 1-10). If the result is greater than a predetermined amount of reported discomfort, steps may be performed to determine the source of the problem. In response to the answer to the follow up question, a treatment plan is put into place addressing the cause of lack of comfort. For example, if the intra-oral sucking vacuum is measured and the pain is associated with a high sucking vacuum from the baby, the positioning and attachment can be altered or a nipple shield may be used. If the pain is not associated with high vacuum the cause may have another cause, for example, infection, vasospasm in the mother or tongue tie in the infant. In all cases, the “test” of measuring intra-oral vacuum may be used to assist in the diagnosis of the problem(s) and the result sends the health care provider or the health care professional down certain treatment option cascades. If the answer is “yes,” a third assessment question is asked.

The third assessment question is something similar or identical to, “Is your milk production enough for your baby?” If the answer is “no,” one or more follow up question would be asked to determine the cause of the insufficient milk. In addition, a study may be performed including an assessment of the number of wet diapers in a 24 hour period, the number of dirty diapers in a 24 hour period and weight gain in a one week period. In response to the answer to the follow up question(s) and study, a treatment plan is put into place addressing the cause of lack of milk. For example, a 24 hour production assessment of maternal milk production, massage, and other treatment options may be performed. If the answer is “yes,” a fourth assessment question is asked.

The fourth assessment question is asked, which is similar or identical to, “Is your baby swallowing and appearing to feed?” If the answer is “no,” one or more follow up question would be asked to determine the cause of the lack of swallowing. For example, if the answer reveals that the mother is not confident that the baby is swallowing or feeding enough, a study may be made of the baby's intake over a 24 hour period. In response to the answer to the follow up question and study, a treatment plan is put into place addressing the swallowing problem.

These four categories comprise the more critical elements relating to a healthy breastfeeding relationship between the mother and baby. As shown in the FIGURE, the assessment 20 may include one or both of clinical assessments as well as objective tests, in some cases.

No score in the above assessment categories means that help is clearly needed. The order of assessment questions, while not critical, is preferred in the order set forth, which ensures key points are always foremost and problems are addressed early. Prompt, appropriate, well-planned assistance saves breastfeeding and reduces conflicting advice from less focused assessments and subjective methods.

An embodiment of the method of the invention may further include determinations, observation and/or testing directed to six identified critical breastfeeding areas of assessment 42. These include, referring to FIG. 1, breastfeeding behavior or function related to the infant tongue 30, the mother's nipple and breast 32, attachment position 34, milk ejection reflex 36, attachment 38, and respiration 40. As shown in the FIGURE, the assessment 42 may include one or both of clinical assessments as well as objective tests.

Should the initial tool 20 reveal some breastfeeding problem, a second phase of the test may include investigation related to assessment related to behavior, physiology and/or function related to assessment areas 30-40. So, in a general form, any person can perform a breastfeeding assessment using the tool 20 described herein, which, if a result of the assessment indicates a problem, negative score or the like a second set of inquiries may be made related to the

To those skilled in the art to which this invention pertains, the above-described preferred embodiment may be subject to change or modification. Such change or modification can be carried out without departing from the scope of the invention, which is intended to be limited only by the scope of the appended claims.

What is claimed is:

1. A method of assessing the quality of breastfeeding, comprising:
determining the contentment of a breastfeeding infant and/or mother;
determining the comfort of the breastfeeding infant and/or mother;
determining if the mother is generating sufficient milk; and
determining if the infant is swallowing appropriately.

2. A method of assessing the health of a breastfeeding relationship between a baby and mother, comprising:
selecting three or all of: determining the contentment of a breastfeeding infant and/or mother, determining the comfort of the breastfeeding infant and/or mother, determining if the mother is generating sufficient milk, and determining if the infant is swallowing appropriately; and
developing and executing a treatment plan based on said determining steps.

3. A method of assessing the quality of breastfeeding, comprising:
making a first assessment related to the contentment of a breastfeeding infant and/or mother, and assigning a score to the result of the first assessment;
making a second assessment related to comfort of the breastfeeding infant and/or mother if the score to the first assessment is positive, and assigning a score to the result of the second assessment;
making a third assessment related to whether the mother is generating sufficient milk if the score to the second assessment is positive, and assigning a score to the result of the third assessment;
making a fourth assessment related to whether the infant is swallowing appropriately if the score to the third assessment is positive, and assigning a score to the result of the fourth assessment; and
formulating and executing a treatment plan if any of the scores are not positive.

4. A method according to any of claims 1 and 2, wherein the result of the determining step is a yes or no assessment.

5. A method of assessing the quality of breastfeeding, comprising:
making a first assessment related to the contentment of a breastfeeding infant and/or mother, and assigning a score to the result of the first assessment;
making a second assessment related to comfort of the breastfeeding infant and/or mother if the score to the first assessment is positive, and assigning a score to the result of the second assessment;
making a third assessment related to whether the mother is generating sufficient milk if the score to the second assessment is positive, and assigning a score to the result of the third assessment;
making a fourth assessment related to whether the infant is swallowing appropriately if the score to the third assessment is positive, and assigning a score to the result of the fourth assessment;
performing a second set of inquiries related to one or more of the infant tongue 30, the mother's nipple and breast 32, attachment position 34, milk ejection reflex 36, attachment 38, and respiration 40; and
formulating and executing a treatment plan based on the result of the second set of inquiries.
